(19) United States
(12) Patent Application Publication
(10) Pub. No.: US 2008/0003566 A1
(43) Pub. Date: Jan. 3, 2008

(54) Molecular Analysis

(76) Inventor: David Vaux, Oxford (GB)

Correspondence Address: BELL & ASSOCIATES, 201 WARREN DRIVE, SAN FRANCISCO, CA, 94131, US

(21) Appl. No.: 10/587,387

(22) PCT Filed: Jan. 26, 2005
(86) PCT No.: PCT/GB05/00244

§ 371 Date: Aug. 10, 2007

(30) Foreign Application Priority Data
Jan. 26, 2004 (GB) 0401639.0
Jun. 18, 2004 (GB) 0412758.5

(51) Int. Cl. C12Q 1/68; C12Q 1/00

(52) U.S. Cl. 435/6; 435/317.1

(57) ABSTRACT

The present invention relates to a method of determining the sequence and/or occurrence frequency of a number of variable gene inserts from a gene library, which inserts exhibit a desired specific characteristic, wherein each variable gene insert is flanked (5′) and (3′) by known sequences, the method comprising; selecting the number of inserts by their ability to exhibit the desired specific characteristic, conducting polymerase chain reaction to amplify the selected number of variable gene inserts to produce components of a mixed PCR product, ligating the components of the mixed PCR product to produce a concatenated sequence and sequencing or determining the occurrence of the gene inserts in the concatenated sequence.

The present invention relates to a method of determining the sequence and/or occurrence frequency of a number of variable gene inserts from a gene library, which inserts exhibit a desired specific characteristic, wherein each variable gene insert is flanked 5′ and 3′ by known sequences, the method comprising; selecting the number of inserts by their ability to exhibit the desired specific characteristic, conducting polymerase chain reaction to amplify the selected number of variable gene inserts to produce components of a mixed PCR product, ligating the components of the mixed PCR product to produce a concatenated sequence and sequencing or determining the occurrence of the gene inserts in the concatenated sequence.

### BACKGROUND TO THE INVENTION

Peptide phage display is a prototypical version of directed in vitro molecular evolution of a large combinatorial library by sequential rounds of physical selection and enrichment. Peptide phage display selection methods have established themselves as powerful tools for the identification of short linear peptide mimetics of many ligand classes. Variant techniques have also been developed extending this methodology to selection from large libraries of oligonucleotides (either random or constrained), translation-arrested ribosomes, phage-displayed binding proteins (of which single chain fragments of immunoglobulin is the largest group) and so on.

A major limitation of all these methods is that the complexity and composition of the selection-evolved sublibrary is assessed by analysis of a very small sample drawn at random from this sublibrary. In consequence, enrichment is deemed to have been achieved when a very few, or more usually one, sequence dominates the sample. This may be acceptable when the evolution is directed by a simple target with one or very few binding sites, but severely limits the method if the target is complex.

The outcome of repeated rounds of selection from a large random peptide phage display library (typically beginning with>10⁹ different phage) is a reduced complexity sub-library enriched for sequences showing specific affinity for the selection matrix. Typically such a sub-library may contain 10³-10⁴ different phage, but in most published studies the outcome of phage panning is assessed by sequencing<20 independent clones.

The problems inherent in directed evolution with a complex target have already been recognised in the field; a theoretical analysis has been presented (Vant Hull et al (1998) J Mol Biol 278 579-597) and a partial practical solution suggested (Messmer et al (2000) J Mol Biol 296 821-832). This “iterative panning” solution relies on completing multiple rounds of directed evolution until a single ‘winning’ sequence emerges. This sequence is then prepared as a synthetic peptide and used in the blocking solution during a repeat of the whole experiment (ie a further set of selections on new target material). Binding of the first class of peptides is now blocked and a second ‘winning’ sequence is selected. This process can be continued indefinitely, each entire round generating one new binding sequence. The method is very slow, very expensive and probably impossible for many key complex biological target materials, since it relies on a large supply of functionally homogenous target material (tumour tissue, infected cells etc) for many rounds of selection.

The present invention addresses the problems identified in the prior art.

The present invention is described with reference to the Figures, in which;

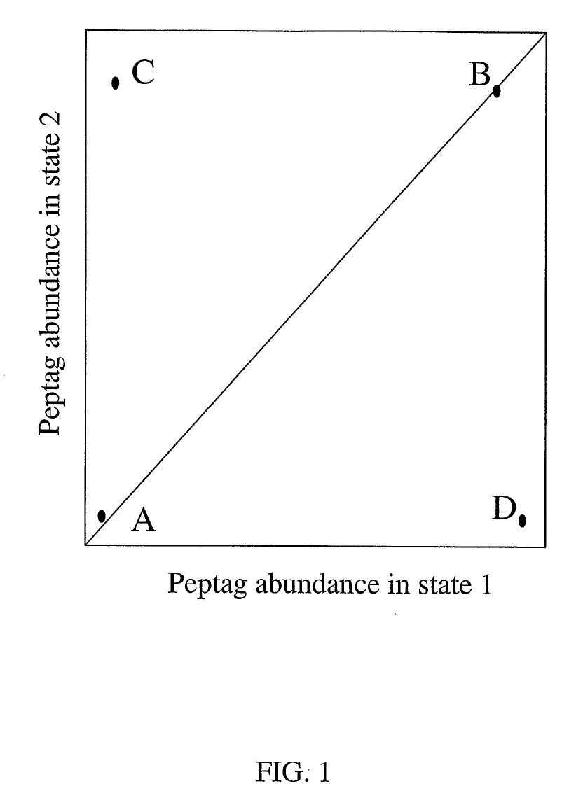

FIG. 1 shows a peptag comparison between two complex samples

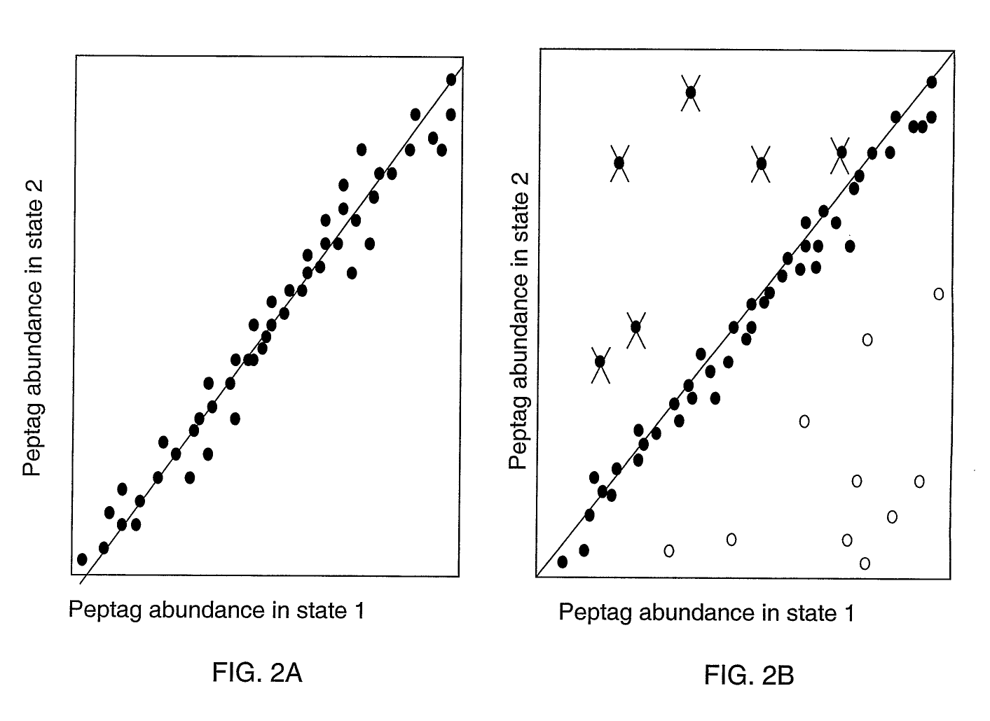

FIGS. 2A and 2B show a peptag comparison between two alternate complex samples

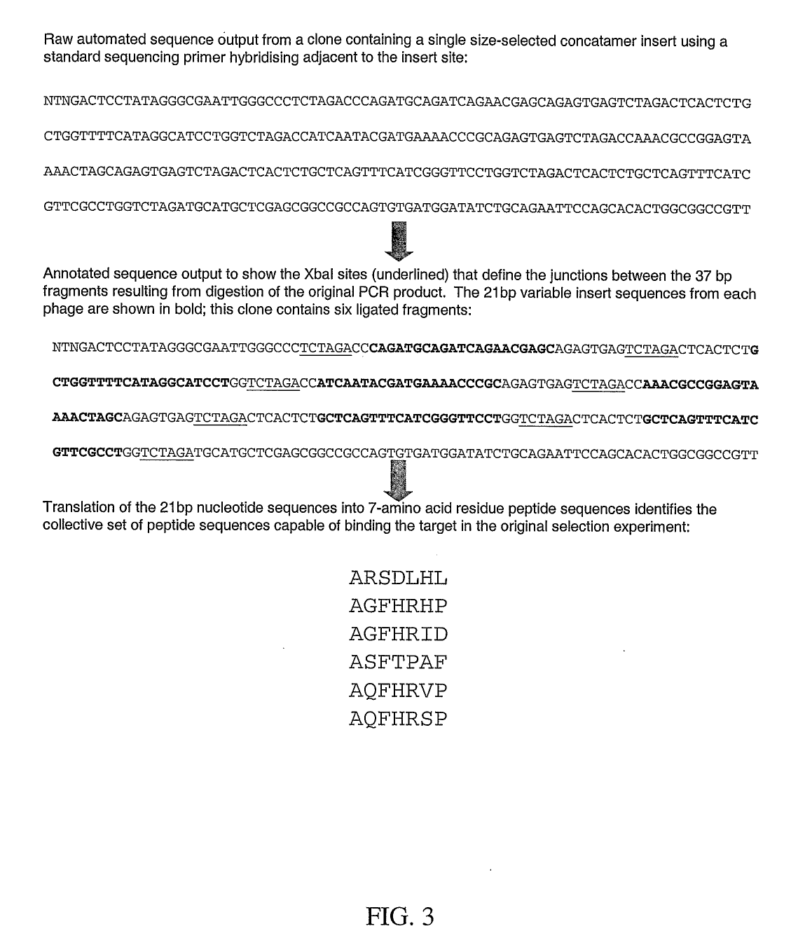

FIG. 3 shows a principle of concatamer data extraction.

### THE PRESENT INVENTION

The present invention provides a method of determining the sequence and/or occurrence frequency of a number of variable gene inserts selected from a gene library, which inserts exhibit a desired specific characteristic and wherein each variable gene insert is flanked 5′ and 3′ by known sequences, the method comprising;

selecting the inserts by their ability to exhibit the desired specific characteristic, conducting polymerase chain reaction to amplify the variable gene inserts to produce components of a mixed PCR product;

ligating the components of the mixed PCR product to produce a concatenated sequence; and

sequencing or determining the occurrence of the gene inserts in the concatenated sequence.

In accordance with the invention, the method can be used for determining the sequence of a number of variable gene inserts or for determining the occurrence frequency of a number of gene inserts from a gene library. A starting gene library generally contains a number of random synthetic sequence fragments representing a wide diversity of all possible peptide sequences of a given length. The invention is independent of the library size, the insert length, and the presence or otherwise of post-translational modifications such as disulphide crosslinks or phosphorylation.

The invention as set out herein and described in claim 1 can be carried out on a random gene library (a starting gene library) or on a gene library which has been treated or selected in some way (i.e. that is pre-selected). Such selection can include positive or negative selection steps.

Preferably the library comprises expressed peptide sequence which are linked with a nucleic acid representation of this peptide sequence. This linkage can be biological or chemical.

An example of a biological linkage would be a peptide sequence encoded in the gene for a phage structural protein displayed as a fused protein fragment on that structural protein on the relevant phage. A gene library containing biological linkages would preferably be a peptide phage display library or a library containing engineered expression of protein associated with, or expressed by, any of, but not limited to, bacteria, yeast, insect cells or mammalian cells. A gene library containing biological linkages would most preferably be a peptide phage display library.

An example of a chemical linkage would be crosslinking of a synthetic peptide with an oligonucleotide that encodes it.

In the present invention, the polymerase chain reaction is conducted using primers complementary to the known sequences 5′ and 3′ to the variable inserts. Further, between the step of ligating the components of the mixed PCR product to produce a concatenated sequence and sequencing or determining the occurrence frequency of the gene inserts, it is preferable to subclone the size-selected concatenated products into a convenient vector for production of plasmid DNA suitable for automated sequencing.

The methodology may include the sequence analysis component of serial analysis of gene expression (SAGE) as described in WO 97/10363 or WO 02/010438 which are hereby incorporated by reference in their entireties.

The present invention relates to a random gene library (of any size, including a large library). The number of variable gene units is selected (or evolved) by identifying those gene inserts which exhibit a desired specific characteristic. This selection may involve one or more cycles of physical selection and amplification to generate a sub-library whose gene inserts encode sequences that share some desired specific characteristics, such as a physical or biological activity. Alternatively, the selection may be of gene inserts which do not exhibit a specific characteristic e.g. do not bind to a particular protein.

In order to determine the number of variable gene according to the present invention, one or more rounds of selection are carried out. These rounds select gene inserts which exhibit a desired specific characteristic. These rounds of selection may reduce the different number of gene inserts from around 10¹¹ to 10⁶ by one round; to 10⁴ by the next round. Each round may be selecting for the same or for a different desired specific characteristic. During any round, it is possible to introduce additional selection criteria or to “block” any binding by the presence of, for example, one or more gene, amino acid or protein sequences which may be present in the library.

The invention provides a simple and economical way of sequencing the relevant variable parts of the gene encoding the phage code protein from a large number (preferably all) of the phage in the selected sub-library. This is in contrast to the prior art which only determined the sequence of a tiny and potentially unrepresentative sub-set of the library. Furthermore, the present invention is a large-scale unbiased analysis without plaque selection or phage DNA purification from selected plaques. The method is achieved by using a polymerase chain reaction with unique primers lying just 5′ and just 3′ to the variable insert in the gene encoding phage coat protein. The reaction is carried out on pooled phage DNA isolated from an aliquot of the library without plaque purification and therefore contains proportional representation amplification of all variable regions in the library or sub-library.

The benefit of the present invention is that each variable region will have an abundance in the double strand DNA product that is proportional to the abundance of that insert sequence amongst the phage in the selected sub-library. The sub-library is the selected number of variable gene inserts on which PCR is carried out according to the present invention. When the components of the mixed PCR product have been prepared, they are ligated to produce a concatenated sequence. It may be useful to digest the components of the mixed PCR product with a very infrequently cutting restriction endonuclease before ligation to produce concatenated sequences. Following production of the concatenated sequences, they may be size selected to around 1.5 kb or below, such as 500-800 base pairs before being cloned into a convenient plasmid. Subsequent sequencing of such inserts generates greater than 30 variable insert sequences per sequencing lane.

The length of each variable gene insert is preferably from 18 to 24 nucleotides or from 18 to 36 nucleotides.

Known software that is used to automatically strip the joining sequences out of continuous DNA sequence to identify and then tabulate the di-tags during serial analysis of ligand-selected peptide display sub-libraries can be used to generate abundance histograms for all the insert sequences identified. FIG. 3 of Example 1 illustrates a continuous DNA sequence, with the variable inserts highlighted between the joining sequences. This figure only shows six peptide sequences, but this method can be extended to collect a peptide list with hundreds of members from which a histogram of peptide tag frequency can be derived. If the target is complex, then a simple consensus may not emerge and the histogram may have multiple small peaks.

The present invention allows the easy analysis of many (often all) of the variable inserts present in the gene library population. This permits the evolution of the selection process to be followed much more accurately. It also ensures that consensus matrix-binding sequences are identified both earlier and more accurately. Also important is that the method of the present invention overcomes the problems of clonal dominance due to the emergence of a single family of binding sequences which prevents analysis of interactions on complex matrices.

The present invention allows the rapid and complete identification of all linear or cysteine cyclised peptides that exhibit a specific behaviour permitting gene selection (either positive or negative). It is also applicable to the classification of all antibody epitopes in a complex humoral response to a pathogen.

The specific behaviour/characteristic permitting gene selection (also described herein as a specific characteristic permitting gene selection) may be any, including the fact that the gene encodes a particular protein which binds to another protein in question.

In addition the gene may encode a particular protein which binds to any target molecule. The target molecule may be organic or inorganic. Alternatively, the gene may encode a protein sequence which only occurs in one state of tissue in comparison with the same tissue in a different state. For example, normal versus tumour tissue, infected versus non-infected tissue, wild type versus mutant, healthy versus oxidatively damaged, healthy versus ischaemic, or occurrence during a particular time zone which is absent at an alternative time zone.

The essence of the proposed invention is a method based on concatenation of short PCR products for efficient sequencing; this permits the analysis of hundreds if not thousands of sequences corresponding to peptides selected at each round of a target-directed evolution from a large combinatorial library. In its simplest form this method reveals multiple sequence families as they are enriched by selection; a single series of enrichment experiments generates frequency histograms for all emergent classes of selected sequence. In this way, multiple binding sites on a complex target are identified without using the time-consuming and expensive iterative panning approach.

The present invention can be utilised in various ways. For example, differential panning on two states (normal versus tumour tissue; infected versus non-infected; wild-type versus mutant; healthy versus oxidatively damaged; healthy versus ischaemic, etc) together with frequency histogram generation on large insert numbers at each round of panning offers a new type of information. The frequency histograms of the two independent panning experiments are compared (in a manner analogous to comparing two SAGE tag profiles, or the microarray binding data from the mRNA samples). This can be done using a simple 2D plot as illustrated by FIG. 1. In FIG. 1, a peptag at point A is of similar low frequency in both data sets while a peptag at point B is of similar high frequency in both data sets (in either case, whatever has changed between state 1 and state 2 does not involve the protein recognised by peptags A or B). A peptag at C recognises a protein that is of low abundance in state 1 but much more highly expressed in state 2. The peptag at D is the reverse; it binds a protein that is abundant in state 1 but much reduced in state 2.

FIG. 2 is a 2D plot with a larger number of peptides. FIG. 2A shows a comparison with no significant change in peptag binding between state 1 and state 2. FIG. 2B shows a comparison of both state 1 increased (white dot) and state 2 increased (dot with cross through) peptags, as well as many with unchanged binding (black spot). This identifies peptide binders that are state independent (ie lying along the diagonal on the two state plot) as well as binders that are enriched in one state or the other. This already enriches the data obtained very substantially. However, by adding a third dimension that identifies the time of appearance of a given sequence during the multiple rounds of panning an entirely new type of information emerges. It is now possible to perform cluster analysis on points that lie off the diagonal within this 3-D space to identify groups of weak signals that together offer a discriminant measure between the two states. To take a specific example, this approach could be used to search for small groups of peptide mimetics that can together discriminate between normal and tumour tissue in a way that could not be achieved by analysis of binding of any single peptide.

A further advantage is that multiple states can be analysed and therefore peptides can be identified that are uniformly unchanged under many states. It is also possible to group those changing in a particular pattern, or altered in only one state, therefore creating a fingerprint for a certain type of state change. The present invention could be utilised to obtain a characteristic fingerprint of a complex target. This is achieved by obtaining large numbers of sequences from sub-libraries, which are created in the early rounds of selection and before one or a small number of sequences are dominating the process. A dataset consisting of a list of binding sequences found in the sub-library, together with the frequency of their occurrence, is regarded as a protein-based equivalent to the nucleic acid-based information derived from a microarray experiment, providing a list of those mRNAs present in a complex sample, together with their abundance. This approach has a number of advantages over the approaches that identify target sequences directly. For example, since the selection is carried out in the protein domain it is sensitive to post-translational modifications in a way that is not possible with nucleic acid microarray approaches. Thus, this approach can be used to compare complex surface glycosylation patterns, or differential phosphorylation, between samples that have each been used to generate an independent dataset. Such pair-wise comparisons can include normal versus transformed cells, normal versus infected cells, normal versus hypoxic cells, normal versus genetically modified cells or cells at different stages of the cell cycle or differentiation programme.

The present invention also relates to a determination of the sequence and/or occurrence frequency of a number of variable gene inserts from a gene library obtained by a method according to the method of the present invention.

The present invention allows characterisation and quantitation of the target molecule responsible for the gene selection or the specific characteristic permitting gene selection. The invention, when used on a known target can be used to select two or more independent peptides that bind the target molecule. Simple competition ELISA permits the selection of pairs of non-competing peptides capable of binding the ligand simultaneously. Such pairs are good candidates for target detection based upon fluorescence resonant energy transfer (FRET) if one peptide was attached to a donor fluorophore and the other peptide was attached to an appropriate acceptor fluorophore. A mixture of two such probes in solution in the absence of the target would have fluorescent spectral properties characteristic of the mixture, whereas addition of the target molecule would recruit donor and acceptor fluorophore into molecular proximity by binding the two different peptides. The outcome is a target concentration-dependent shift in the spectral properties to include a FRET signal. Such a FRET signal is then measured as a way to quantitate target molecules in solution. Since the peptide and fluorophore combined may have a molecular mass below 1.5 kDa, this approach has the potential to overcome the steric limitations that make paired immunoglobulin assays (such as ‘sandwich’ ELISAs) unsuitable for some important small molecules such as cytokines and interleukins.

An example of this process is exemplified by preparing fluorophore conjugates of synthetic peptides that mimic antigen and bind to the antigen combining sites on a particular immunoglobulin. In this case, a mixture of donor fluorophore-peptide and acceptor fluorophore-peptide would show FRET only when intact immunoglobulin was added. Addition of an irrelevant immunoglobulin would give no FRET signal because no pairs of peptide would be immobilised in molecular proximity. Furthermore the experiment can be used to demonstrate the need for two peptide attachment sites since addition of the same number of antigen binding sites but in the form of monovalent F(ab) fragments would result in no FRET signal being detected. [This process has been christened ADLIRP, standing for analyte detection by ligand immobilisation of resonant proteins/probes.]

Thus, one feature of the invention is the use of a molecular selection technique to isolate multiple binding probes from a complex random library. This step can be achieved by monoclonal antibody production (although the requirement for two binding sites on the ligand would make this sterically difficult), or, more usefully, by peptide phage display methods or by SELEX methods to select aptamers.

The invention works equally well if the two probes were peptides (e.g. the 7 or 12 mers identified by peptide phage display) or aptamers (derived by RNA SELEX) or any combination of these or other specific ligand binding molecules. The key requirement is for two non-competitive binding sites on the analyte under study.

The invention works utilising FRET because of the very tight distance dependence of fluorescent resonance energy transfer (resonance transfer proportional to 6th power of fluorophore separation). Donor and acceptor fluorophores attached to separate probes can co-exist in solution without showing FRET, but if the two probes bind to separate sites on a common ligand then the donor and acceptor fluorophores are held in close proximity and FRET results.

This aspect of the invention is made possible by the use of selection methods designed to facilitate the identification of two probes with the required specificity. In one embodiment this selection method is peptide phage display using the separately described “Phage-SAGE” technique to produce a histogram of candidate sequences and their isolation frequency. Alternatives would include SELEX methods for aptamer selection or any small molecule screen. For example, one probe might be a known interacting small molecule that could be labelled with a fluorophore. It would then only be necessary to find one other probe capable of independent binding to the analyte to make a solution FRET pair.

Since the peptide and fluorophore combined may have a molecular mass below 1.5 kDa, this approach has the advantage of overcoming the steric limitations that make paired immunoglobulin assays unsuitable for some important small molecules such as cytokines and interleukins. Further advantages of this approach is that the two probe method operates in dilute solution (without dimerisation giving false FRET signals) and the measured parameter is the appearance of a FRET signal. Linker geometry does not have to be optimised and both negative and positive control mixtures can be easily prepared to quality control both sensitivity and dynamic range within each assay. Furthermore, in principle it should be possible to multiplex the system by mixing more than one probe pair in a single solution provided that the spectral properties of the pairs are sufficiently separated to prevent cross-talk. In an extension of the method, a third binding probe that competes with either the donor probe or the acceptor probe (i.e. overlapping binding sites) can be used to refine the specificity of the assay even further (to detect the amount of a cytokine and the presence or absence of a polymorphism, for example).

Other embodiments of the invention allow for the rapid selection of paired binding probes to any desired and available analyte. If the probe labels are not fluorophores interacting by FRET but oligonucleotides interacting by base pairing, for example, a PCR based amplification step can be set up.

There are additional approaches that could utilise the present invention in order to characterise the target molecule.

For example a peptag could be synthesised as a peptag—GST fusion protein and incubated with a lysate of the material in which the target is known to be present. The resulting complex could then be retrieved using glutathione agarose beads washed and subjected to mass spectroscopy to identify the bound target protein. An alternative, would be to synthesise a peptide containing the peptag followed by a biotin mimic sequence and incubated with a lysate of the material in which the target is present. The resulting complex could then be retrieved using streptavidin beads. Mass spectroscopy could then be used to measure the mass of the intact protein and/or proteolytic fragment fingerprint mass spectroscopic mapping.

The present invention is now described with reference to the following, non-limiting, examples:

### EXAMPLE 1

### Method of Determining Sequence and Occurrence Frequency of a Number of Variable Gene Inserts that Bind to a Target Molecule

The variable gene inserts were selected from a seven amino acid peptide phage display library, with an input of 2×10¹¹ phage particles. Therefore all possible seven residue sequences were represented on average more than 50 times.

The target was prepared by immobilising and purifying IgG immunoglobulin of a monoclonal antibody raised against a known peptide antigen (AEFHRWSSYMVWHK).

Four rounds of selection were performed on the library, followed by elution, amplification and re-selection. This produced a sub-library.

Unselective PCR was performed on the variable region encoding the individual peptides from all phage in the sublibrary.

The PCR was performed using 22 bp primers that are known to hybridise to known sequences just 5′ and just ′3 to the variable insert sequence and each encoding the restriction endonuclease Xbal site, that does not occur in the variable region. The initial product was 73 bp and had known 5′ and 3′ ends, but variable regions carrying representative sequence information.

This material was digested to completion with Xbal which produced 37 bp fragments with Xbal sticky ends. These fragments were purified away from released termini by polyacrylamide gel electrophoresis. The fragments were then ligated using DNA ligase.

The resulting concatamers were again subjected to polyacrylamide electrophoresis. This allowed selection of fragments of approximately 500-800 bp which were purified from the gel.

The concatamer fragments were ligated into a prepared Xbal cut, dephosphorylated vector. The resulting ligation products were used to transform competent bacteria. Subsequently colonies were selected as a source of plasmid DNA. Sequencing was performed on the plasmid DNA using standard primers flanking the vector multiple cloning site.

The result was a linear DNA sequence of repeated 37 bp fragments between Xbal sites, with each repeat containing one 21 bp variable sequence encoding one of the binding peptides in the selected sub-library. These 21 bp nucleotide sequences were translated to give 7 amino acid peptide sequences which were entered into a database. This data extraction is illustrated by FIG. 3. Analysis of over 50 sequences obtained from linear DNA sequences revealed that 98% of the sequences contain the consensus AxFHRxx. This sequence also being present in the target antigen sequence.

1. A method of determining the sequence and/or occurrence frequency of a number of variable gene inserts from a gene library which inserts exhibit a desired specific characteristic, wherein each variable gene insert is flanked 5′ and 3′ adjacent known sequences, the method comprising:
selecting the number of inserts by their ability to exhibit the desired specific characteristic, conducting polymerase chain reaction to amplify the selected number of variable gene inserts to produce components of a mixed PCR product;
litigating the components of the mixed PCR product to produce a concatenated sequence; and
sequencing or determining the occurrence of the gene inserts in the concatenated sequence.

2. A method as claimed in claim 1 wherein the gene library is a peptide phage display library.

3-14. (canceled)

15. The method as claimed in claim 1, wherein the components of the mixed PCR product are digested with a restriction endonuclease before ligation to produce the concatenated sequence.

16. The method as claimed in claim 1, wherein the concatenated sequence is cloned into a plasmid before sequencing.

17. The method as claimed in claim 16, wherein the concatenated sequence is size selected to from about 500 to about 1.5 kilobases in length before cloning into the plasmid.

18. The method as claimed in claim 2, wherein the length of each variable gene inserts is from about 18 to about 36 nucleotides.

19. The method as claimed in claim 18, wherein the specific characteristic is one or more of protein binding or occurrence only in one state of tissue in a comparison with the same tissue in a different state.

20. The method as claimed in claim 2, wherein the number of variable gene inserts are from phage which do not exhibit a specific characteristic.

21. The method as claimed in claim 1, wherein selecting the number of inserts by their ability to exhibit the desired specific characteristic comprises two or more rounds of selection based on the ability of the variable gene inserts to exhibit the desired characteristic.

22. The method as claimed in claim 1, wherein when more than one round of selection by the ability of the insert to exhibit the desired characteristic is carried out, then the different rounds of selection may be of more than one desired specific characteristic.

23. The method as claimed in claim 1, further comprising characterisation of a target molecule.

24. The method as claimed in claim 1, further comprising quantitation of a target molecule.

25. The method as claimed in claim 23, wherein characterisation comprises using fluorescence resonant energy transfer.

26. The method as claimed in claim 24, wherein quantitation comprises using fluorescence resonant energy transfer.

27. A dataset of a sub-library resulting from the method of claim 1.

28. A determination of the sequence or occurrence frequency of a number of variable gene inserts from a gene library obtained by the method of claim 1.
